(19) United States
(12) Patent Application Publication
(10) Pub. No.: US 2009/0275721 A1
(43) Pub. Date: Nov. 5, 2009

(54) Coating Surfaces

(76) Inventors: Matthew Cooper, Cambridge (GB); Xin Li, Cambridge (GB)

Correspondence Address: MORGAN LEWIS & BOCKIUS LLP, 1111 PENNSYLVANIA AVENUE NW, WASHINGTON, DC, 20004, US

(21) Appl. No.: 12/225,107

(22) PCT Filed: Mar. 16, 2007
(86) PCT No.: PCT/GB2007/000914

§ 371 Date: Sep. 15, 2008

(30) Foreign Application Priority Data
Mar. 16, 2006 (GB) 0605302.9

(51) Int. Cl. B05D 3/00; C07K 17/08

(52) U.S. Cl. 527/207; 427/2.13

(57) ABSTRACT

Disclosed is a method of attaching, indirectly, a member of a specific binding pair (or sbp) to a surface, the method comprising the steps of: (a) contacting the surface with a solution, preferably an aqueous solution, of a polymer, having side chains according to the formula X-Y-Z-R, wherein X is a spacer group; Y is a sulphur, selenium or tellurium atom; Z is a sulphur, selenium or tellurium atom, any of which may be bonded to one or two oxygen atoms; and wherein R is any suitable moiety such that -Z-R constitutes a leaving group; such that at least some of the -Z-R groups are displaced and the polymer becomes bound to the surface by X-Y groups; and (b) contacting a polymer-coated surface resulting from step (a) with a solution, preferably an aqueous solution, comprising an sbp member, so as to cause the polymer to react with the sbp member, so as to attach the sbp member, indirectly, to the surface.

### FIELD OF THE INVENTION

This invention relates to a method of indirectly attaching a molecule, such as a member of a specific binding pair, via an intermediate polymeric layer, to a surface especially, but not exclusively, a metal surface.

### BACKGROUND OF THE INVENTION

Biosensors typically utilise a biological recognition molecule, immobilised on a solid support, such as a gold, platinum or silver surface. The biological recognition molecule may be, for example, an oligo- or polynucleotide, but more usually comprises a polypeptide, such as an antibody or antigen-binding fragment of antibody or variant (e.g. scFv, F(ab), F(ab)′₂ domain antibody [“dAb”], or multimers thereof).

The solid surface is desirably substantially inert, so as to avoid damaging the biological recognition molecule and to avoid affecting the sample applied to the biosensor. However, the inert nature of the solid support means that it is difficult to attach the biological recognition molecule directly to the solid support. Moreover, such direct attachment (i) limits the total amount of biological recognition molecule that can be attached, and (ii) tends to cause at least some loss of biological recognition activity e.g. due to denaturation of the polypeptide. Additionally, reagents or samples to which the metal surface may be exposed during synthesis and/or use of the biosensor may cause corrosion of the metal surface. For these and other reasons, it has become conventional to fully or partially coat the solid support with an intermediate layer, the biological recognition molecule then being attached to the intermediate layer rather than directly attached to the solid support.

The intermediate layer typically comprises a self-assembly monolayer (“SAM”, e.g. U.S. Pat. No. 5,242,828), a dendrimer (Langmuir 2005, 21(5), 1858-65; Langmuir 2004, 20(16), 6808-6817), polymer brush (Adv. Colloid Interface Sci. 2003, 100-102, 205-65) or other polymer.

Attachment of the intermediate layer to the solid support generally requires the use of fairly reactive compounds and/or organic solvents, which are incompatible with most biological recognition molecules, especially polypeptides, causing denaturation, loss of activity, etc. For this reason, the intermediate layer must first be attached to the solid support, and the reactive compounds removed by thorough washing, before the relatively delicate biological recognition molecule can be attached (using far gentler conditions and reagents) to the intermediate layer.

This two step attachment process suffers from several disadvantages: waste of biological recognition molecule in the washing step; relatively large amounts of the biological recognition molecule are required; and recycling of the biological recognition molecule or extended incubation in contact with the intermediate layer or solid support are necessary, both of which increase the risk of denaturation of the recognition molecule.

International Patent Application No. PCT/GB2005/003455, unpublished at the date of filing of the present application, discloses certain polymers and their use in the formation of an intermediate layer on solid substrates, to which can be attached a biological recognition molecule. The polymers comprise covalently bound side chains of the formula X-Y-Z-R, wherein X is a spacer group, Y is a sulphur, selenium or tellurium atom; Z is a sulphur, selenium or tellurium atom, any of which may be bonded to one or two oxygen atoms; and wherein R is any suitable moiety such that -Z-R constitutes a leaving group.

Typically at least some of the -Z-R groups are displaced, such that the polymer becomes bound to a surface, and remaining unreacted -Z-R groups are then reacted with a biological recognition molecule, such as a receptor or antibody, to immobilise it to the surface.

### SUMMARY OF THE INVENTION

In a first aspect the invention provides a method of attaching, indirectly, a member of a specific binding pair (or sbp) to a surface, the method comprising the steps of: (a) contacting the surface with a solution, preferably an aqueous solution, of a polymer, having side chains according to the formula X-Y-Z-R, wherein X is a spacer group; Y is a sulphur, selenium or tellurium atom; Z is a sulphur, selenium or tellurium atom, any of which may be bonded to one or two oxygen atoms; and wherein R is any suitable moiety such that -Z-R constitutes a leaving group; such that at least some of the -Z-R groups are displaced and the polymer becomes bound to the surface by X-Y- groups; and (b) contacting a polymer-coated surface resulting from step (a) with a solution, preferably an aqueous solution, comprising an sbp member, so as to cause the polymer to react with the sbp member, so as to attach the sbp member, indirectly, to the surface.

Those skilled in the art will understand that the order of the steps can be reversed. Accordingly, in a second aspect the invention provides a method of attaching, indirectly, an sbp member to a surface, the method comprising the steps of: (a) contacting, in solution, preferably in aqueous solution, an sbp and a polymer containing side chains according to the formula X-Y-Z-R as aforesaid, so as to cause the sbp member to become attached to the polymer; and (b) contacting an a solution, preferably an aqueous solution, of sbp member/polymer complex resulting from step (a) with the surface so as to cause displacement of -Z-R groups from at least some X-Y-Z-R side chains of the polymer to attach to the sbp member/polymer complex, indirectly, to the surface.

Equally, it is also possible to perform the method of the first or second aspects of the invention in substantially a single step. Accordingly, in a third aspect the invention provides a method of attaching, indirectly, an sbp member to a surface, the method comprising the step of contacting the surface with an a solution, preferably an aqueous solution, comprising a polymer having side chains according to the formula X-Y-Z-R as aforesaid, and an sbp member, so as to form a complex of polymer/sbp member bound to the surface.

In this aspect of the invention the solution of the polymer and the solution of the sbp member may be mixed, in the presence of the surface. The use of the term “single step” is intended to indicate that, during at least part of the process, the surface is in contact with a solution, (preferably an aqueous solution) which comprises both the polymer and the sbp member, and that, during this part of the process, the surface has not yet been fully occupied by polymer, such that there are still binding sites on the surface available for the polymer to attach to.

Also in this aspect of the invention, the process may desirably be performed in a single reaction vessel and conveniently be performed by causing both the polymer and the sbp member to be present, simultaneously, in solution, preferably in aqueous solution. The single reaction vessel may be, for example, the well of a microtitre plate, an Eppendorf tube or other container, flask, or a flow cell or conduit associated with analytic or synthetic (e.g. SPR) apparatus or the like. In an especially preferred embodiment of the invention, essentially all the steps may be performed in a single reaction vessel, including even an initial derivatization of the polymer, (preferably a dextran polymer), in which X-Y-Z-R side chains are formed on the polymer, carboxyl or other reactive groups (if any) present on the polymer may be activated (conveniently by use of conventional EDC/NHS chemistry), an sbp member coupled to the polymer, and the polymer bound to a solid surface, all of the steps conveniently taking place in aqueous solution. (The order of the steps is not critical, except that the side chains must be added to the polymer before it is deposited on a solid surface; and, if used for this purpose, carboxyl or other reactive groups must be activated before coupling the sbp member).

Conveniently, but not necessarily, the sbp member may be attached to the polymer by reaction with the -Z-R groups of at least some of the -X-Y-Z-R side chains. This may result in the formation of an -X-Y-Z-R₁ side chain, wherein R₁ is the member of the sbp. More generally the sbp member may be attached to the polymer by reaction with a reactive group (preferably a charged group) which may or may not form part of the X-Y-Z-R side chain. Preferred reactive groups include hydroxy, carboxy, epoxy and amino groups. If desired, charged or other reactive groups (such as hydroxy, carboxy, epoxy and amine groups) may be introduced into the polymer by reaction with the X-Y-Z-R side chains. Alternatively (or additionally), the charged or other reactive groups may be present in other side chains and/or on the main chain of the polymer. The charged or other reactive groups may be introduced into the polymer before, during or after attachment of the polymer to the surface.

The polymers of use in the present invention are such that relatively gentle reaction conditions and reagents may be used to attach the polymer to the surface so that, if desired, the sbp member can be bound to the polymer before the polymer is attached to the surface, or the polymer and sbp member can be bound to each other substantially simultaneously with attachment of the polymer to the surface without encountering problems of inactivation or denaturation of the sbp member.

The ability to use solutions of polymer and sbp member means that, in some embodiments, the attachment of the sbp to the surface via the intervening polymer can essentially be effected in a single step, which is convenient, efficient and economical. Aqueous solutions are generally preferred. This is because most sbp members will normally be compatible with water and aqueous solutions should therefore substantially preserve the desired binding activity of the sbp, whilst other solvents may well cause denaturation of polypeptide sbp members. In addition, aqueous solutions are generally easier to handle, are non-volatile and do not require use of specialised apparatus.

For present purposes, a solution may be thought of as aqueous if water constitutes 50% v/v or more of the liquid present in a solution. Preferably the solution will be such that water constitutes 80% or more of the liquid present, more preferably 90% or more, and most preferably 95% or more of the liquid present in the solution.

A further advantage is that the present invention eliminates the necessity of attaching an sbp member to a polymer coated surface. Attachment of the sbp member to a surface has hitherto been the rate limiting step, since reaction with surfaces is limited by considerations of mass transport. In contrast, by performing reactions with reagents free in a solution, the reaction is much more rapid, as both reagents are free to diffuse (see, for example, Berg & von Hippel 1985 Ann. Rev. Biophys. Biophys. Chem. 14, 131-160) with a greater number of degrees of freedom than when one of the reagents is already attached to the surface.

This facilitates, for example, the use of neutral polymers to form an intermediate layer on a surface, to which an sbp member may be attached. In contrast, in order to address low reaction rates, the prior art (exemplified by U.S. Pat. No. 5,436,161) suggests coating surfaces with polymers having “charged groups, for bringing about a concentration of biomolecules carrying an opposite charge to that of said charged groups” (i.e. using polymers of opposite charge to that on the sbp member to be attached to the polymer).

Finally, the ability to use aqueous solutions confers much greater flexibility, ease of use and simplifies or even abolishes the need for washing steps etc.

Preferred features of the polymers of use in the methods of the present invention are generally as described in PCT/GB2005/003455, a copy of which is attached hereto.

Favourably R is a moiety such that the conjugate acid HZR has a pKa of less than 8 and preferably less than 6, more preferably less than 4.

Favoured values for Y are S and Se of which S is particularly apt. Favoured values for Z include S, SO and SO₂ of which S and SO₂ are particularly apt.

Preferred values for -Y-Z- include S—S and S—SO₂ of which S—S is particularly preferred.

Apt values for R when Z is a S, Se or Te atom, and preferably a S atom, include unsaturated groups conjugated to electron withdrawing groups, aromatic groups and heteroaromatic groups and electrophilic groups. Suitable electron withdrawing groups include lower alkyloxycarbonyl, nitrite, nitro, lower alkylsulphonyl, trifluoromethyl and the like. Suitable aromatic groups include optionally substituted phenyl where there are up to three substituents selected from nitro, trifluoromethyl, nitrile, lower alkyloxycarbonyl, or other electron withdrawing groups. (Throughout the present specification the term “lower” is used to mean containing up to 6 carbon atoms, more aptly 1-3 carbon atoms and favourably 1 or 2 carbon atoms, unless the context clearly dictates otherwise). Suitable heteraromatic groups include those of 5- or 6-membered rings optionally fused to the residue of phenyl ring or a further 5- or 6 membered heteroaromatic ring. Said heteroaromatic group may be optionally substituted by one or two lower alkyl, phenyl, ═O, ═S, trifluoromethyl, nitro or nitrile groups.

Particularly apt groups —S—R include those derived from aromatic thiols and heteraromatic thiols or their thione tautomer. Particularly suitable —S—R groups include those derived from imidazole; pyrrolidine-2-thione; 1,3-imidasolidine-2-thione; 1,2,4-triazoline-3(5)-thione; 1,2,3,4-tetrazoline-5-thione; 2,3-diphenyl-2,3-dehydrotetrazolium-5-thione; N(1)-methyl-mercaptopiperidine; thiomorphyline-2-thione; thiocaprolactam; pyridine-2-thione; pyrimidine-2-thione; 2-thiouracil; 2,4-dithiouracil; 2-thiocytosine; quinoxazoline-2,3-dithione; 1,3-thiazoline-2-thione; 1,3-thiazolidine-2-thione; 1,3-thiazolidine-2-thione-5-one; 1,3,4-thiadiazoline-2,5-dithione; 1,2-oxazolidine-2-thione; benz-1,3-oxazoline-2-thione; 1,3,4-oxadiazoline-2-thione and analogues in which the sulphur is replaced by selenium or tellurium.

A particularly favoured R group is the 2-pyridyl group (2-Py).

A preferred -Z-R group is the —S-2-pyridyl group.

A preferred -Y-Z-R group is the —S—S-2-pyridyl group.

The spacer group -X- will aptly be of the formula -A-B-. The nature of group -A- will depend upon the group in the polymer to which the side chain will be attached. The most common groups of the polymer to which the side chain will be attached are CO₂H, OH and optionally mono lower alkyl substituted NH₂.

Other suitable groups present in the polymer which may be utilised for attaching the side chain include those used for immobilisation in liquid chromatography such as aldehyde, hydrazide, carboxyl, epoxy, vinyl, phenylboronic acid, nitrile-triacetic acid, imidodiacetic acid and the like.

In the case of polymers containing carboxyl groups the side chain may be attached via an ester group —CO—O—B—. Thus the group -A- represents an oxygen atom attached to the residual carboxyl group of the original carboxyl group. In an alternative the side chain may be attached via an amide group where the group -A- is an —NH— group or lower alkyl substituted —NH— group.

In the case of polymers containing hydroxy groups, the side chain may be attached via an acylated hydroxy group —O—CO—B—. Thus the group —O—X- may represent a —O—CO—B—, —O—CO—O—B—, —O—CO—NH—B— or lower alkylated —O—CO—NH—B— group.

In the case of polymers containing primary or secondary amino groups, the side chain may be attached in an analogous manner to the case for hydroxy containing polymers. Thus the group —NH—X- or its lower alkyl substituted derivative may represent —NH—CO—B—, —NH—CO—O—B—, —NH—CO—NH—B— or their lower alkyl substituted derivatives.

The other suitable groups referred to above may be used for attachment of side chains in manners appropriate to their chemistry as will be understood by the skilled chemist.

The group B may be any convenient group such as an alkylene, phenyl or like group which may be unsubstituted or substituted by lower alkyloxy, halo, oxo, trifluoromethyl, nitrile or other group that does not interfere with the formation and use of the -Y-Z-R moiety.

Particularly apt groups B include lower alkylene groups optionally interrupted by an oxygen atom, carboxyl group or carbonyloxy group. Favoured groups include straight chained alkylenyl groups —(CH₂)ₙ— where n is 1, 2, 3 or 4, and is preferably 2.

In conjunction with hydroxy substituted polymers, the spacer group X is aptly of the formula —CO—O—X1- or —CO—NH—X1- where X1 is a lower alkylenyl group. Suitable alkylenyl groups include —CH₂—CH₂—, —CH₂—CH₂—CH₂— and —CH₂—CH₂—CH₂—CH— groups which may optionally be interrupted with a hetero-atom, for example —O—, or substituted (for example by —OH). The —CO—NH—CH₂—CH₂— group is the preferred spacer group.

A particularly preferred value for the -X-Y-Z-R group for attachment to hydroxy group containing polymers is the —CO—NH—CH₂—CH₂—S—S-2-pyridyl group.

Typically, after attachment of the member of the sbp to the polymer, the polymer may contain side chains of the formula -X-Y-Z-R1, where R1 is the member of sbp.

The term “specific binding pair” is used in respect of two molecules which have a specificity for each other so that under normal conditions they bind to each other in preference to binding to other molecules and most aptly to the essential exclusion of others. Suitable binding pairs include antibodies and antigens, ligands and receptors, and complementary nucleotide sequences. One or both members of the pair may be part of a larger molecule, for example the binding domain of the antibody, a section of a nucleotide sequence and the like. Ligands, for example hormones, cancer marker proteins and the like, may be suitable agents for binding to receptors on cell surfaces or the like to determine cells possessing said receptor. Suitable members of pairs may include molecular imprints, aptamers, lectins and the like. Typically the member of the specific binding pair coupled to the polymer will comprise a peptide or a polypeptide.

A particularly favoured member of a specific binding pair is an antibody. A fragment of antibody may be used as long as it possesses the binding fragment such as a Fd, Fv, Fab, F(ab prime)2 and single chain Fv molecules linked to form an antigen binding site.

One member of a specific binding pair may be attached to some of the side chains on the polymer by replacing some of the -Z-R moieties by -Z-R1 moieties where R1 is the residue of the member of a specific binding pair. This may involve reaction with a thiol group naturally present in the member of the specific binding pair, for example a protein. Alternatively an HZ- group could be synthetically added to the member of the binding pair (in a manner which does not prevent the modified member binding with the other member of the pair). For example, hydroxy groups or amino groups could be acylated with an active derivative of mercaptopropionic acid or the like.

The polymer employed in this invention may be a synthetic or natural polymer. It may take the form of a hydrogel, a porous matrix, a gel, a crosslinked polymer, a star polymer, a dendrimer or the like. Such polymers may also be co- or ter-polymers, graft polymers, comb-polymers and the like.

In particular, in one embodiment, the invention encompasses the use of complex polymer structures, in which the receptor moiety R1 is further removed from the surface to which the polymer is coupled. In an example of such an embodiment, a substrate is coated with a complex structure: a relatively short polymer in accordance with the invention is coupled to the substrate by displacement of -Z-R leaving groups from X-Y-Z-R side chains. A relatively long molecule or moiety is attached to the polymer (before or after coupling to the substrate), which relatively long moiety comprises a plurality of biologically or chemically reactive groups for attachment of a receptor, antibody or other member of a specific binding pair. By placing the leaving groups -Z-R at a distal end of the brush or comb polymers, thereby distancing the receptors R1 from the substrate, it may be possible to construct a biocompatible surface coating that is more permeable to both protein and small molecule binding, enabling higher receptor densities on the substrate and/or higher signal:noise ratios in biosensor applications. The relatively long moiety may be attached to the polymer by chemical or enzymatic ligation, or by polymer “grafting”. Thus a matrix may be formed from, for example, copolymers (including comb polymers, alternating copolymers, block copolymers) and terpolymers. Methods of polymerisation are well known to those skilled in the art and representative general teaching of suitable techniques is given by Matyjaszewski & Xia (2001, Chem. Rev. 101, 2921-2990) and Hawker & Wooley (2005, Science 309, 1200-1205).

It is preferable that the polymer used in this invention is hydrophilic, biocompatible and, when present as a coating, is able to resist non-specific binding of analytes and contaminants. Suitable polymers include dextran, hyaluronic acid, sepharose, agarose, nitrocellulose, polyvinylalcohol, partially hydrolysed polyvinylacetate or polymethylmethacrylate, carboxymethyl cellulose, carboxymethyl dextran and the like.

The use of the polymers of this invention can lead to an excellent level of coating of the metal which contributes to the reduction of non-specific binding observed in this invention. This is most easily achieved by using a neutral (uncharged) polymer. The term “neutral” as used herein, is intended to indicate that a polymer does not contain any readily ionisable groups, and therefore will be uncharged in all physiological environments.

Charged polymers according to this invention (for example those derived from polymers containing residual carboxylate groups such as carboxymethylated cellulose derived polymers) can be used to enhance binding of desirable moieties. For present purposes, a “charged” polymer is one which comprises readily ionisable groups, (e.g. —OH, —NH₂, —COOH). Accordingly, under certain conditions of pH or the like, a “charged” polymer may in fact be neutral, because the groups in question are uncharged or because the polymer comprises equal numbers of positive and negative charges, whilst in other conditions, the polymer will carry a net charge, due to ionisation of the readily ionisable groups.

It has been found that particularly good resistance to non-specific binding can occur with the use of neutral (uncharged) polymers of the invention.

Particularly apt derivatisable polymers for use in this invention are those derived from sugar monomeric units. Conveniently the polymer has an average molecular weight in the range 10 KDa-1,000 KDa. Preferably the polymer has an average molecular weight in the range 50 KDa-1,000 KDa. More preferably the polymer has an average molecular weight in the range 100 KDa-1,000 KDa. Most preferably the polymer has an average molecular weight of about 250 KDa to 1,000 KDa.

A preferred derivatisable polymer for use in this invention is dextran. Suitable grades of dextran include T10, T70 and T500. These have average molecular weights of 10, 70 and 500 KDa respectively. T500 Dextran appears to be at or near the optimal average molecular weight.

Without wishing to be bound by any particular theory, the inventors hypothesise that coatings formed from lower molecular weight polymers, such as T10 or T70 Dextran are sub-optimal in terms of their thickness and are too thin to be able to associate with many members of a specific binding pair, whilst T500 Dextran forms thicker coatings and can couple more molecules of a specific binding pair. Additionally, or alternatively, the thicker coating formed by bigger molecular weight polymers are better able to protect the underlying sensor surface and/or prevent non-specific binding.

The surface to be coated will preferably, but not necessarily, be a metal one that has reactivity with chalcogen containing molecules. Such metals include those of groups 10 and 11 and titanium. Favoured metals include gold, silver, platinum, palladium, nickel, chromium, titanium and copper and their alloys of which gold and platinum are most favoured. A preferred metal is gold.

Metal ions may also be employed such as cadmium, ferrous and mercurous ions. An adhesion coat may be present between the substrate and the metal if desired. A preferred adhesion coat comprises titanium.

The surface is conveniently metallic, and may be essentially planar. However, neither of these features is essential. In particular, the surface may be particulate or colloidal. For present purposes, a colloid may be defined as a system in which finely divided particles (typically about 1-1,000 nm Angstroms in mean diameter) are dispersed within a continuous medium in a manner that prevents them from being filtered easily or settled rapidly.

In one embodiment the surface may comprise a colloidal metal. For example, colloidal gold is widely used in the performance of immunoassays and is readily available commercially (e.g. from British Biocell International Limited, Cardiff, UK). The present invention provides a simple and convenient way of attaching antibodies or other molecules to the surface of colloidal gold particles.

The method of the invention may also be used with non-metallic surfaces. A particular example of such a non-metallic surface includes semi-conductors and quantum dots (also known as “semiconductor nanocrystals”). Quantum dots may be made in large numbers by the technique of pyrolytic synthesis and quantum dots made in this way typically comprise cadmium selenide. Quantum dots are useful as artificial fluorophores: they are extremely small, having a diameter in the range of about 10 nm to 300 nm (i.e. less than the wavelength of visible light) and when illuminated with white or ultraviolet light they emit very intense fluorescence.

There are two basic methods of forming a colloid: reduction of larger particles to colloidal size, and condensation of smaller particles (e.g., molecules) into colloidal particles. Some substances (e.g., gelatin or glue) are easily dispersed (in the proper solvent) to form a colloid; this spontaneous dispersion is called peptization. A metal can be dispersed by evaporating it in an electric arc; if the electrodes are immersed in water, colloidal particles of the metal form as the metal vapor cools. A solid (e.g., paint pigment) can be reduced to colloidal particles in a colloid mill, a mechanical device that uses a shearing force to break apart the larger particles. An emulsion is often prepared by homogenization, usually with the addition of an emulsifying agent. The above methods involve breaking down a larger substance into colloidal particles. Condensation of smaller particles to form a colloid usually involves chemical reactions—typically displacement, hydrolysis, or oxidation and reduction.

Derivatisation of colloids in the prior art has been achieved by several different mechanisms:
1. Direct physisorption of receptors to sensitized bare gold colloids—this results in stable preparations of coated colloids, but with low receptor activity due to receptor denaturation and random orientation on the metal surface;
2. Coupling of receptors via intermediate self-assembled monolayer (SAM) layers that passivate the metal surface and provide specific chemical linkages for chemical coupling, but this procedure is technically demanding and involves multiple steps which adds significantly to cost of goods.
3. Coupling of receptors via intermediate amphiphilic polymer layers that are physisorbed to the gold to passivate the metal surface and provide specific chemical linkages for receptor coupling. However, for this approach a highly hydrophobic moiety (normally a polyaromatic) is required to physisorb to the gold. This renders the polymer coating intrinsically less hydrophilic, with less desirable properties for particle dispersion in aqueous matrices, lower activity of biological and biochemical receptors, and higher non-specific binding to matrix components.

The present invention confers an additional advantage when applied to colloids. Colloidal suspensions are very sensitive to changes in their surface chemistry and/or the surrounding environment, either of which can cause large scale aggregation of the colloids and loss of the colloidal structure/dispersion. For example, transferring a colloidal suspension from distilled water into PBS is often sufficient to cause aggregation. Accordingly, a multi-step process of activating the colloidal particles, coupling a polymer to the particles, coupling a receptor to the polymer etc. causes frequent changes in surface chemistry and/or environment of the colloidal particles with a consequent high risk of aggregation. Conversely, the solution phase in situ coupling process of the present invention can be performed in just a single step and therefore minimises the risk of causing aggregation.

In one embodiment the invention encompasses reacting an aqueous solution of a polymer as aforesaid possessing side chains containing -X-Y-Z-R groups, and optionally -X-Y-Z-R1 groups, with a substrate, preferably a metal surface, so that at least some -Z-R groups are displaced and the polymer becomes attached to the metal surface via -X-Y- side chains.

Thus in one favoured aspect, this invention provides a metal surface coated with a hydrophilic polymer which has side chains of the formula -X-Y-Z-R1 wherein X, Y, Z and R1 are as previously defined. Apt, favoured and preferred values for X, Y and Z and combinations of X, Y and Z are as stated hereinbefore.

In the case where the polymer contains both of the preceding side chains, those with -Z-R groups will be displaced preferentially, thus providing an attached polymer which contains -X-Y-Z-R1 groups attached to the metal.

A metal surface is most suitably supported on a more robust substrate layer. This substrate may be of any convenient materials, typically (but not necessarily) suitable for use as a biosensor. In the case of conventional assay, plastics such as polystyrene are used, or glass. Piezoelectric or optical materials may also be used. Glass or quartz are the preferred substrates for optical biosensors, particularly surface plasmon resonance devices. Suitable piezo-electric materials are well known and include quartz, lithium tantalate, gallium arsenide, zinc oxide, polyvinylidene fluoride and the like, but quartz is most suitable.

The substrate may be employed in an active or a passive device for the detection and/or characterisation of a member of a specific binding pair which becomes bound to the other member of the specific binding pair which is present on the side chain attached to the polymer.

In an alternative method -ZR groups may be displaced by groups which contain a reactive moiety to which the member of the specific binding pair can become attached. In such an embodiment the group R1 can be considered as the residue of the specific binding pair which also incorporates a linker group at the point of attachment to the group Z.

Such an alternative method can be used for the attachment of the group R1 via an amino, hydroxy, carboxy or like group. For example, a polymer containing -X-Y-Z-R side chains can be reacted with N-succinimidyl 3-(2-pyridyldithio)propionate (hereinafter, SPDP) which then provides side chains -X-Y—S—CO—O-1-pyrrol-2,5-dione which on reaction with a primary amine within R1 provides a -X-Y—S—CH₂CONHR2 side chain wherein CH₂CONHR2 represents R1.

The apt, favoured and preferred values for the amine of the formula NH₂—Y-Z-R are as set out hereinbefore. Thus, for example a favoured amine for use is that of the formula H₂N—CH₂—CH₂—S—S-2Py.

If desired some of the active leaving groups may be displaced to yield side chains containing -X-Y-Z-R1 moieties as hereinbefore described. Alternatively, such side chains may be introduced after coupling of the polymer to a surface.

Methods of making polymers of use in the present invention are set out in detail in our co-pending application WO 2006/027582.

The present invention may find particular application in the synthesis of biosensing surfaces. In the manufacture of a biosensor it is well known to deposit a layer of metal on a substrate. Thus, for example a thin layer of titanium may be coated onto a substrate such as glass, quartz, plastic or the like. Such adhesion layers are generally formed by vapour deposition and are 0.5-5 nm thick, more usually 1-2.5 nm, for example about 1.5 nm thick. A thicker layer of a metal as hereinbefore described, particularly gold, platinum or silver and preferably gold, is then coated over the adhesion layer, for example by vapour deposition. The thickness of such metal layers depends on the biosensing technique to be employed and may be from about 10-200 nm, more usually about 20-100 nm, for example 35-75 nm thick. For piezoelectric methods the thickness is typically 100-200 nm for example.

The active side chain containing polymer may be bound to the surface of the metal by bringing an aqueous solution of the active side chain polymer into contact with the metal surface. Generally, before contacting the metal surface with the solution of the polymeric agent, the surface is cleaned, for example by washing with ultra-pure water, then with sodium hydroxide and surfactant solution and then more ultra-pure water.

The cleaned surface is then contacted with the aqueous solution of polymeric agent, for example for 3 to 30 minutes, more usually 10-20 minutes. Typically the solution may contain 0.1-10%, for example 0.5 to 7.5%, of polymer containing -X-Y-Z-R and/or -X-Y-Z-R₁ side chains. The contact may be static or the solution may be moved relative to the metal surface. During this time, -Z-R groups are displaced and the polymer becomes bound to the metal via -X-Y- groups. For example, in the case of a polymer containing -X—S—S-2Py groups, the —S-2Py group is displaced and the polymer attached to the metal by -X—S— bonds. After this binding has occurred, any non-chemisorbed polymeric material may be removed by washing with sodium hydroxide. The resulting polymer coated metal is unaffected by acid, base, salts, detergents or cysteine at concentrations likely to be encountered in use in a biosensor.

Since only some of the active leaving groups are displaced by binding to the metal, the metal surface is coated with a layer of polymer which polymer retains some -X-Y-Z-R side chains as hereinbefore described.

It is believed that the coating thus achieved covers a higher proportion of the metal surface than what has been achieved by prior art methods which have not employed good leaving groups -Z-R.

The -X-Y-Z-R group can be reduced to a free Y group, (-X-YH); for example where Y is sulfur, reduction to a sulfhydryl group (—SH); by a reducing agent, such as dithiothreitol (DTT), and then reacted with an agent such as SPDP or a sulphated analogue. The resulting polymer containing -X-Y—S—CH₂—CO—O—N═(COCH₂CH₂CO) side chains (or other N-hydroxysuccinamide analogues with a SO₄²⁻ salt) may then be reacted with amino groups present in R1 moieties or derivatised R1 moieties, for example proteins and particularly antibodies. If R1 is a small molecule (for example a ligand that binds to a receptor to be analysed) it may be linked by reaction with a sulphydryl or amino group it possesses or it may be derivatised to include such a group. Thus, for example a hydroxy group may be esterified with 3-mercaptopropionic acid or glycine or the like. Similarly, a carboxy group may be esterified with NH₂CHCH₂OH, HSCH₂CH₂OH or the like. Alternatively, a cross reactive analogue of a natural ligand can be used which contains a sulphydryl or amino group or a derivatised hydroxy or carboxy group or the like.

In an alternative process the metal surface may be contacted with a solution of a polymer containing both -X-Y-Z-R and -X-Y-Z-R1 side chains. The polymer becomes bound to the surface by displacement of -Z-R groups leaving -X-Y-Z-R1 side chains in place, since -Z-R is a better leaving group than -Z-R1.

In a further alternative process the metal surface may be contacted with a solution of a polymer containing -X-Y-Z-R side chains. The polymer becomes bound to the metal surface by displacement of -Z-R groups, and other groups present in the polymer before modification are converted to reactive side chains.

Any residual active disulphide groups may be inactivated (capped) by exposure to an aqueous solution of, for example, cysteine, in 100 mM borate buffer at pH 8.5.

It will be appreciated that, in a favoured aspect, this invention provides a biosensor comprising (i) a substrate; (ii) a layer of metal on a surface of said substrate; (iii) a polymer attached to said metal by side chains of the formula -X-Y-; and (iv) said polymer also having side chains of the formula -X-Y-Z-R1; wherein X, Y, Z and R1 are as hereinbefore defined.

Preferably, but not necessarily, the X-Y groups in the two types of side chain are the same. The groups X, Y, Z and R1 are aptly, favourably and preferably as hereinbefore described. The substrate and metal will aptly, favourably and preferably be as hereinbefore described. The polymer will aptly, favourably and preferably be as hereinbefore described.

The person skilled in the art will appreciate that the present invention provides, in effect, a number of different routes to the desired end point of attaching a member of a specific binding pair to a polymer deposited on a surface.
Route 1: activation of the polymer in solution, attachment of the sbp member to the activated polymer in solution, and deposition of the resulting polymer/sbp member complex onto the surface. The activation of the polymer and attachment of the sbp member may be performed in successive steps or substantially simultaneously, in a single step (e.g. in a single reaction container).
Route 2: activation of the polymer in solution (preferably aqueous), deposition of the activated polymer on the surface, and attachment of the sbp member to the deposited polymer (from aqueous solution).
Route 3: deposition of the polymer on the surface then contacting the deposited polymer with an aqueous solution of the activating agent(s) and the sbp member (either successively or simultaneously).
Route 4: attachment of the sbp to the polymer, in solution, (preferably aqueous solution), possibly to preexisting reactive groups present in the polymer (e.g. —COOH groups), activation of the polymer and deposition on the surface by displacement of at least some -Z-R groups, typically from an aqueous solution phase.

Each of these routes is within the scope of the invention.

The inventors have found that, following activation of the polymer into a state in which there are reactive groups available for reaction with the sbp member, it is desirable to wait before contacting the polymer with the sbp member to be coupled to the polymer. A convenient delay before exposure of the polymer to the sbp member is in the range 5-60 minutes, preferably 10-50 minutes, more preferably 10-45 minutes and most preferably 10-40 minutes. It is to be emphasised that such a delay is not essential, but has been found to confer optimal performance.

The present invention will now be described by way of illustrative examples and with reference to the accompanying drawings, in which:

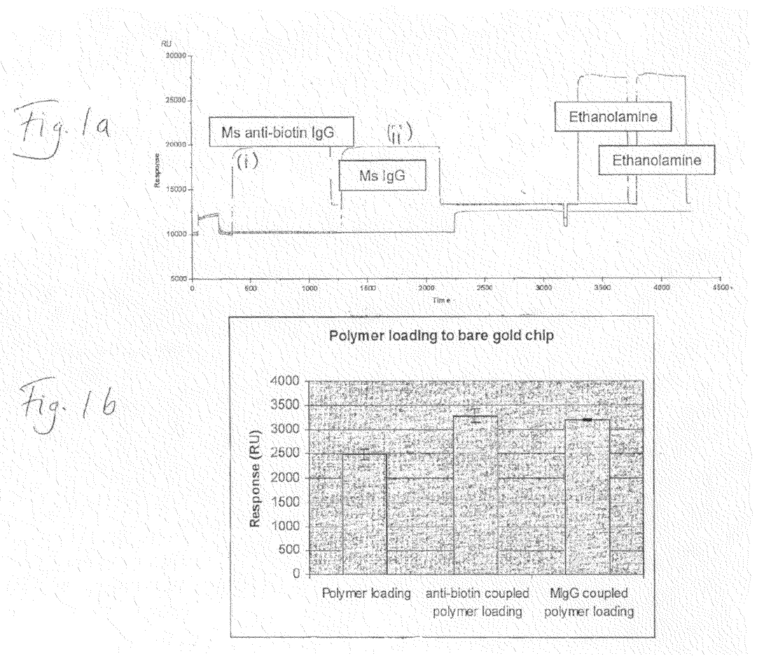

FIG. 1 shows the SPR traces (response against time in seconds) obtained when a gold biosensor surface was treated with an aqueous solution of an active polymer (T70-CMD-PDEA) that had previously been coupled in free solution to either mouse anti-biotin IgG(i) or to a control mouse IgG(ii);

FIG. 1b presents the same data in bar chart from, showing an increased SPR response following coating of the gold surface with the anti-biotin or control IgG-coupled polymers;

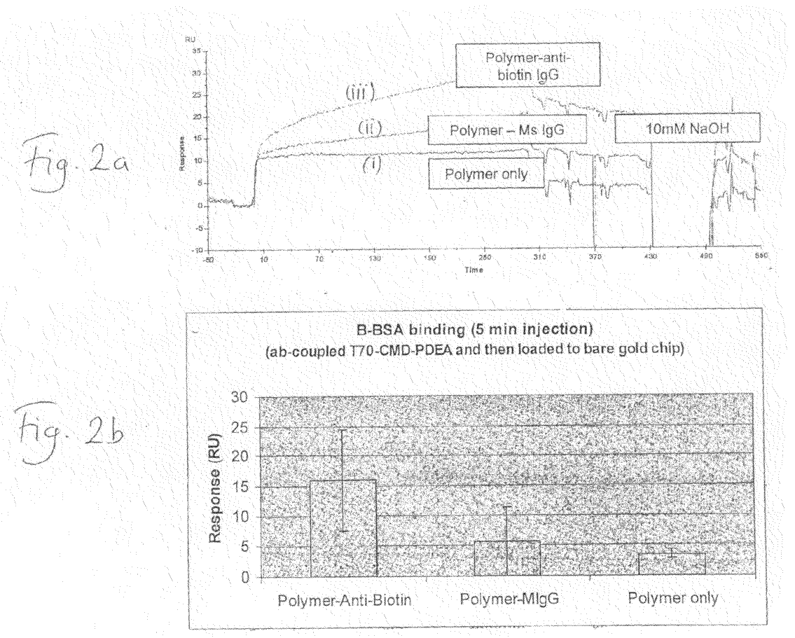

FIG. 2a shows the SPR traces (arbitrary response units against time, in seconds) following exposure of biotinylated BSA to a biosensor surface coated with (i) polymer only; (ii) polymer coupled to control mouse IgG; or (iii) polymer coupled to mouse anti-biotin IgG.

FIG. 2b shows the data from FIG. 2a presented as a bar chart.

FIG. 3 shows the net SPR trace (response against time, in seconds) from FIG. 2a for binding of biotinylated BSA to the surface coated with polymer coupled to mouse anti-biotin IgG, after subtraction of the level of non-specific binding (as represented by binding to the surface coated with polymer only.

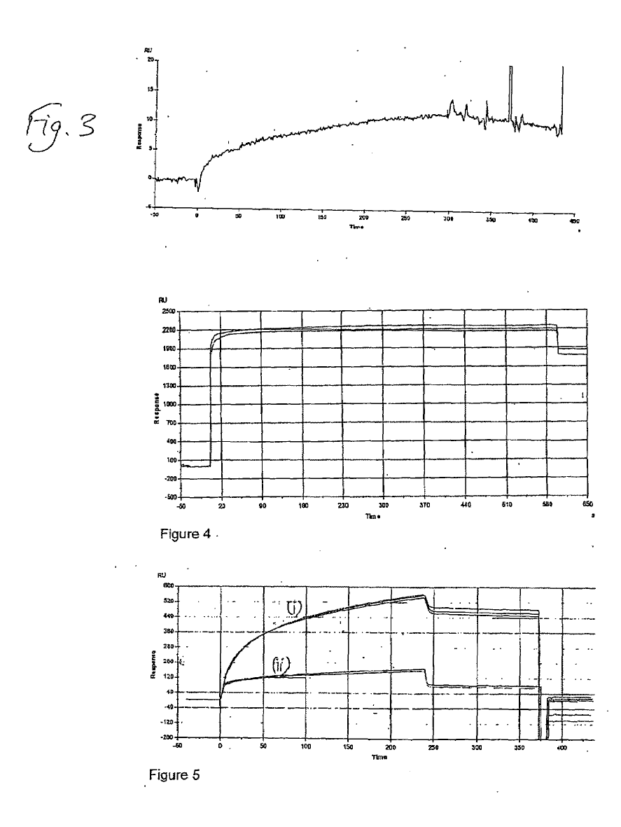

FIG. 4 shows the SPR trace (response against time, in seconds) obtained when loading a gold surface with an aqueous solution of a dextran-based thiosulfone polymer coupled to either bovine or human serum albumin (BSA or HSA), and

FIG. 5 shows the SPR trace obtained when the respective coated surfaces were exposed to an anti-HSA antibody. More of the antibody was bound to the HSA/polymer-coated surface (i) than to the BSA/polymer-coated surface (ii).

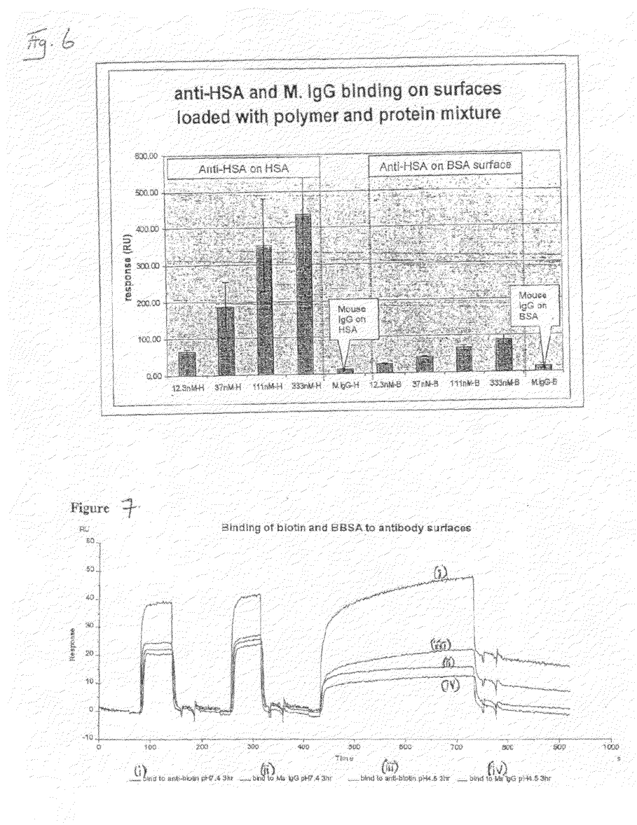

FIG. 6 is a bar chart showing the SPR response (in arbitrary response units) when anti-HAS antibody or a control mouse IgG was contacted with a sensor surface coated with a dextran thiosulfone-based polymer coupled to HAS (left hand portion of chart) or to BSA (right hand portion of chart). The numbers at the bottom of the chart indicate the concentration of anti-HAS antibody used.

FIG. 7 shows SPR traces for surfaces coated at pH 7.4 with polymer coupled to anti-biotin or a control mouse IgG (traces (i) and (ii) respectively) or coated at pH 4.5 (traces (iii) and (iv) respectively), and the resulting response when the surfaces are contacted with biotin or biotinylated BSA.

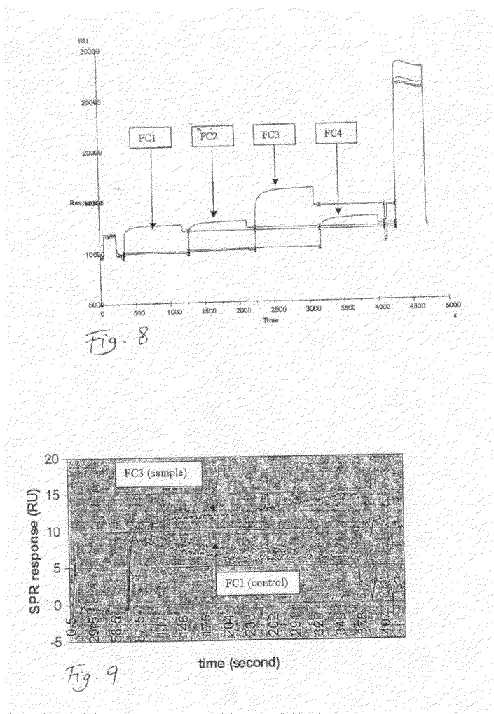

FIG. 8 is a graph of SPR response (arbitrary units) against time (seconds) showing the response of four different sensors treated in different ways, as explained in Example 5.

FIG. 9 is a graph of SPR response (arbitrary units) against time (seconds) for four different sensors when exposed to biotinylated BSA.

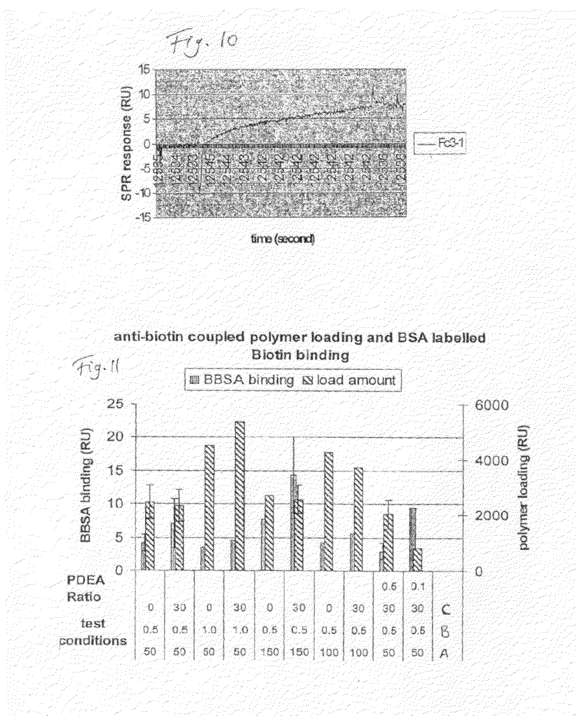

FIG. 10 is a graph showing the net response of one of the traces shown in FIG. 9, after subtraction of the response of the control sensor.

FIGS. 11-13 are a bar charts showing response level (arbitrary units) for various different experimental conditions.

### EXAMPLES

### Example 1

### Materials

1. Akubio Ti/Au chip (Lot. no. 2805) (Material AF 45 (9.82×11.89 mm), 0.30 mm thick from Glass Perfection, Camb. UK (Corning No. 2 glass, clean and scratch free). Metal coating: 1.5 nm Ti+47 nm Au e-beam vapour deposited.
2. Biacore 2000 SPR system
3. Degassed PBS
4. Milli Q water
5. 1% TritonX-100/100 mM NaOH solution (0.22 μm filtered)
6. 10 mM NaOAc buffer pH 4.5
7. EDC (400 mM,)
8. NHS (100 mM,)
9. Ethanolamine (1 M, pH 8.5)
10. Mouse monoclonal anti-biotin (Jackson ImmunoResearch Laboratories, Inc. Pennsylvania, USA)
11. Mouse IgG, 2 mg/ml in PBS (Jackson ImmunoResearch)
12. Biotin-BSA, 1 mg/ml in PBS,
13. Dextran-T70-CMD-PDEA

### Synthesis of Dextran-COOH-PDEA

### Intermediate

1. Synthesis of (2-(pyridinyldithio)ethaneamine (PDEA)—Used in Step 3 Below.

Alrithiol-2 (4.41 g, 20 mmol) was dissolved in 20 ml of methanol and 0.8 ml of acetic acid. Into the solution was added 2-aminoethanethiol hydrochloride (1.14 g, 10 mmol) in 10 ml of methanol. The reaction mixture was stirred for 2 days, then concentrated in vacuo to give a yellow oil. The yellow oil was then thoroughly washed by vigorously stirring with diethyl ether. The clear yellow supernatant was decanted off, and the residue dissolved in methanol (10 ml). To the resultant solution was added 50 ml of ether, and the precipitate separated by filtration. This procedure was repeated three times, then the resultant final solid purified by recrystallisation (methanol/diethyl ether) to give a pale white solid (1.62 g).

### 2. Carboxymethyl Dextran

1.6 grams of dextran T70 (about 10 mmol glucose unit) was dissolved in 4M NaOH aqueous solution (25 ml) (4 g NaOH in 25 ml water). To this, was added a solution of MCA (monochloroacetic acid, 7.56 g) and NA₂CO₃ (4.24 g) in 20 ml water. The resulting solution was stirred at 90-100° C. for 3 hours. Then it was acidified with concentrated HCl aqueous solution with a cooling bath of ice-water to pH3.0, and purified by dialysis against distilled water until pH7.0 was obtained, and dried by lyophilization to give a white fluffy solid.

Functionality Degree=0.64 (Functionality degree is the % of substituted COOH of total OH in unsubstituted glucose) IR: 1733.7 cm⁻¹, 1635.4 cm⁻¹.

### 3. Dextran-COOH-PDEA

500 mg of Dextran T70-COOH from Step 2 above (D.S. 0.64) was dissolved in 16 ml of anhydrous DMSO at 60° C. The resulting clear solution was then cooled to room temperature, and to it, were added 383 mg of HOBt (2.5 mmol) and 0.78 ml of diisopropyl carbodiimide (DIC). The mixture was stirred at room temperature for 10 minutes, and then 3 ml of pyridine and 0.55 grams of PDEA (2-(pyridinyldithio)ethaneamine) was added. The resulting mixture was stirred at room temperature overnight. It was then dropped into 50 ml of acetone. To the resulting mixture were added 20 ml of ether and 200 ml of petroleum ether. The resulting precipitates were then collected by filtration, further washed with acetone 5 times, and dried under high vacuum to give a yellow powder 508.3 mg.

NMR data for the pyridinyl group/dextran were obtained as follows:

¹H NMR (300 MHz, D₂O) δ_{H} 8.41 (1H, br), 7.81 (2H, br), 7.30 (1H, br). Microanalysis (%): N=3.7 and 3.7, functionality: 1.32 mmol/g.

### Formation and Measurement of Polymer/SBP Complex

A 1 mg/ml solution of T70-CMD-PDEA polymer in PBS, 50 μg/ml Mouse IgG [or Mouse monoclonal anti-biotin] in PBS, 200/50 mM EDC/NHS were mixed together in aqueous solution in a glass bijou tube and incubated at room temperature for 1 h. A bare gold SPR sensor chip was docked in a Biacore 2000 System, which was then primed with running buffer. Under a constant flow of running buffer at 10 μl/min, the gold surface was cleaned with a 3 min injection of 1% Triton X-100/100 mM NaOH. Then under a constant flow of running buffer at 5 μl/min 70 μl of the anti-biotin coupled polymer was injected over FlowCell 3, then 70 μl of the Mouse IgG coupled polymer was injected over FlowCell 2. 70 μl of untreated 1 mg/ml polymer (T70-CMD-PDEA in PBS) was injected over FlowCell 1. The flow of running buffer was increased to 100 μl/min, then 50 μl of 10 mM NaOH was injected over all three flow cells, followed by two injections of 70 μl of 1 M Ethanolamine pH8.5 on flow cells 2 and 3 only.

The SPR traces for flow cell 3 (trace i) and flow cell 2 (trace ii) are shown in FIG. 1a. In both traces a clear increase in response is apparent after injection of the respective polymers, indicating deposition of the polymer on the gold surface. This is also apparent in FIG. 1b, which plots the data as a bar chart.

Running buffer (PBS) was then passed at a flow rate of 10 μl/min over Flow Cells 1,2,3. Biotinylated BSA (BBSA) at 10 μg/ml in PBS was then injected over Flow cells 1,2,3 for 5 min, followed by a regeneration pulse of 10 mM NaOH for 1 min. The resulting traces are shown in FIG. 2a. Trace (i) is that for the gold chip coated with polymer only. Trace (ii) is for the surface coated with polymer coupled to control mouse IgG, and trace (iii) is that for the surface coated with polymer coupled to mouse anti-biotin. Again, the data are also presented as a bar chart (FIG. 2b). It is apparent that there is a higher response for the surface coated with polymer coupled to anti-biotin antibody. This indicates greater capture of the biotinylated BSA on the surface, suggesting that the anti-biotin antibody is functional and specifically binds the biotin moiety. FIG. 3 shows the ‘net’ trace derived from FIG. 2a (i.e. binding of BBSA to the anti-biotin coupled polymer, after substraction of the non-specific binding).

### Results

### Example 2

### Summary of Solution Conjugation of Proteins to Thiosulphone Active Polymer, and Assay Results

### Synthesis of Dextran (T70) Thiosulphone

### 1. Intermediate

### 4-Nitrophenyl Carbonated Dextran (Used in Step 4 Below).

To a solution of dextran T70 (1.6 grams, 29.6 mmol OH) in 18 ml of anhydrous DMSO and 16 ml of anhydrous pyridine, were added 4-nitrophenyl chloroformate (800 mg, 4 mmol) and catalytic amounts of DMAP(N,N-Dimethylamino-4-pyridine) with stirring at 0° C. (external cooling bath). The reaction mixture was stirred at this temperature for 1 hour, then at room temperature for a further hour. The solution was slowly added into a mixture of methanol and ether (1:1) (150 ml) with vigorous stirring. The precipitates formed were collected with filtration, and washed with the same solvent mixture 3 times. The collected white solid was dried under high vacuum to give 1.54 grams of white powder.

### 2. Sodium Methanethiosulfonate

A mixture of sodium methanesulfonate (1 gram, 9.8 mmol) and sulfur (312 mg, 9.75 mmol) in methanol (60 ml) was heated under reflux for 3 hours by which point all sulfur had dissolved. The mixture was then filtered, and the filtrate was concentrated in vacuo to dry to give a white solid.

¹H NMR (300 MHz, D₂O)_{H} 3.375 (3H, s)

IR: 1322.9 cm⁻¹, 1201.5 cm⁻¹, 1097.3 cm⁻¹, 977.7 cm⁻¹, 769.3 cm⁻¹

### 3. S-(2-Aminoethyl) Methanethiosulfonate Hydrobromide

2 grams of sodium methanethiosulfonate from Step 2 above (15 mmol) and 2 grams of 2-bromoethylamine hydrobromide (10 mmol) were dissolved in 50 ml of methanol. The resulting solution was then refluxed for 3 hours, and it was concentrated in vacuo to dry. The residue was suspended in 30 ml of acetonitrile, and the precipitates were filtered off. The filtrate was concentrated in vacuo to form a yellow sticky oil. To the residue, was added a mixture of acetonitrile and diethylether (2.5 ml/2.5 ml) and the mixture was stirred vigorously overnight to give a yellow pasty solid. The remaining liquid was poured off and the solid was suspended in above solvent mixture (2.5 ml/2.5 ml) and triturated. The mixture was filtered, and the solid was washed with the above solvent mixture, and ether to give an off-white solid, and dried in vacuum with P₂O₅ to give 1.45 gram powder.

¹H NMR (300 MHz, D₂O)_{H} 3.497 (5H, m), 3.371 (2H, t, J=17), 4.240 (2H, br)

¹³C NMR (300 MHz, D₂O)_{C} 50.994 (CH₃), 41.016 (CH₂), 34.149 (CH₂)

IR: 1309.4 cm⁻¹, 1132.0 cm⁻¹

4. Methanethiosulfonated dextran T70

600 mg of 4-nitrophenylcarbonated dextran T70 from Step 1 above was dissolved in 12 ml of DMSO and 3 ml pyridine. To this solution were added 200 μl of NMM and 300 mg of S-(2-aminoethyl) methanethiosulfonate hydrobromide from Step 3. The resulting solution, was stirred at room temperature overnight, and was precipitated in a solvent mixture of methanol and ether (1:1) (75 ml) and the precipitates was collected by filtration and washed with the same solvent 3 times, and dried under high vacuum to give a white powder.

Microanalysis (%): N=0.66 and 0.79, S=4.00 and 4.10

Functionality: 0.58 mmol/g

### Storage of Polymers

All polymers were stored as lyophilised powders at −20° C. under nitrogen. Between 0.1 and 2% w/v solutions of polymer in ultra-pure water were prepared by gentle shaking at room temperature until the solution was visibly clear, then they were centrifuged at 14000 rpm in a Genofuge 16M bench top centrifuge to precipitate any undissolved material. The supernatant was then carefully removed with a pipette and stored at 4° C. until required.

Method
1. Dextran T70-thiosulfone was prepared as described above, and incubated with BSA in PBS for 1 hr at room temperature (control surface), or
2. with HSA in PBS for 1 hr at room temperature (active surface).
3. The complexes 1 and 2 above were loaded into Flow cell1 and 2 respectively of the Biacore 2000 onto a bare gold sensor surface, at a flow rate of 10 μl/min, and then washed for capping for 5 minutes with 50 mM cystein in 100 mM NaOAc pH4.5 and 1 M NaCl. For all assays, PBS was used as running buffer. The results are shown in FIG. 4 below. As can be seen from the Figure, both polymers bound to the surface to achieve very similar levels of loading.
4. 111 nm Anti-HSA was allowed to flow across both cells at 5 μl/min in triplicate followed by wash with 10 mM HCl for regeneration, at a flow rate of 40 μl/min for 5/40 minutes and the response measured as shown in FIG. 5 below. Trace (i) shows the results for the HSA-coupled polymer and trace (ii) the results for the BSA-coupled polymer. As hoped, the antibody bound more effectively to the HSA-coupled polymer coated surface.

Using the HSA surface, with the BSA surface as a control, different concentrations of anti HSA (12.3 nM, 37 nM, 111 nM and 333 nM) and control mouse IgG (111 nM), at a flow rate of 10 ml/min for 4 minutes were contacted in series. The surfaces were regenerated with 10 mM HC 1 at 40 μl/min for 10/40 minutes and the response measured. The results are shown in FIG. 6 below.

FIG. 6 shows that for more of the anti-HSA antibody bound to the HAS-coupled polymer coated surface than to the BSA-coupled polymer coated surface. For example, at an antibody concentration of 37 nM, the SPR response for the BSA surface was just under 50 RU, whilst the response for the HSA surface was nearly 200 RU, about 4-fold greater. For both surfaces the amount of irrelevant control mouse IgG bound was insignificant, indicating that binding of the anti-HAS antibody was primarily antigen-specific.

### Example 3

### Deposition of T70CMD-PDEA-antibiotin and T70CMD-PDEA-Mouse IgG onto SPR Bare Sold in Flow Mode, and on Binding of Biotin and Biotinylated BSA to the Resulting Surfaces

### Equipment

1) Biacore 2000 SPR system;
2) Akubio Ti/Au chip (Material AF 45, 9.82×11.89 mm, 0.30 mm thick from Glass Perfection, Camb. UK). Metal coating: 1.5 nm Ti+47 nm Au e-beam vapour deposited.

### Reagents

1) Au—SPR chips were used;
2) Biacore SPR 2000.
3) 0.5% SDS, 0.22 μm filtered (for Desorbing step 2)
4) 50 mM glycine, pH 9.5, 0.22 μm filtered (for Desorbing step 3)
5) MilliQ water, 0.22 μm filtered, degassed
6) Running buffer:
   a. PBS, 0.22 μm-filtered & degassed
7) Coupling buffer for mouse anti-biotin and mouse IgG:
   a. 10% PBS diluted just before use
   b. 100 mM NaOAc buffer, pH4.5
8) Ethanolamine 1M, pH 8.5
9) Mouse anti-Biotin (Jackson ImmunoResearch, 1.2 mg/ml)
   a. 833 μl of 1.2 mg/ml stock added to 167 μl of PBS pH7.4, to give 1 mg/ml;
   b. 50 μl of 1 mg/ml anti-biotin solution was added to 950 μl of PBS pH7.4, to give 50 μg/ml;
10) Control mouse IgG (Jackson ImmunoResearch, 5.5 mg/ml)
   a. 182 μl of 5.5 mg/ml stock added to 818 μl of PBS pH7.4, to give 1 mg/ml;
   b. 50 μl of 1 mg/ml mouse IgG solution was added to 950 μl of PBS pH7.4, to give 50 μg/ml;
11) Antibody coupled active polymer solutions:
   a. 1 mg/ml T70CMD (Carboxymethyl dextran)-PDEA, 50 μg/ml anti-biotin, 200 mM EDC and 50 mM NHS solution in 10% PBS. To prepare this solution mixture were combined:
      i. 0.5 ml 2 mg/mil polymer in 10% PBS;
      ii. 50 μl 1 mg/ml anti-biotin in PBS;
      iii. 225 μl 888 mM EDC (dilute 170.45 mg EDC in 1 ml 10% PBS);
      iv. 225 μl 222 mM NHS (dilute 25.6 mg NHS in 1 ml 10% PBS);
   b. 1 mg/ml T70CMD-PDEA, 50 μg/ml mouse IgG, 200 mM EDC and 50 mM NHS solution in 10% PBS. To prepare this solution mixture were combined:
      i. 0.5 ml 2 mg/ml polymer in 10% PBS;
      ii. 50 μl 1 mg/ml mouse IgG in PBS;
      iii. 225 μl 888 mM EDC (dilute 170.45 mg EDC in 1 ml 10% PBS);
      iv. 225 μl 1222 mM NHS (dilute 25.6 mg NHS in 1 ml 10% PBS);
   c. 1 mg/ml T70CMD-PDEA, 50 μg/ml anti-biotin, 200 mM EDC and 50 mM NHS solution in 10 mM NaOAc buffer pH4.5. To prepare this solution mixture were combined:
      i. 0.5 ml 2 mg/ml polymer in 10 mM NaOAc buffer pH4.5;
      ii. 50 μl 1 mg/ml antibiotin in PBS;
      iii. 225 μl 888 mM EDC (dilute 170.45 mg EDC in 1 ml 10 mM NaOAc buffer pH4.5);
      iv. 225 μl 222 mM NHS (dilute 25.6 mg NHS in 1 ml 10 mM NaOAc buffer pH4.5);
   d. 1 mg/ml T70CMD-PDEA, 50 μg/ml mouse IgG, 200 mM EDC and 50 mM NHS solution in 10 mM NaOAc buffer pH4.5. To prepare this solution mixture were combined:
      i. 0.5 ml 2 mg/ml polymer in 10 mM NaOAc buffer pH4.5;
      ii. 50 μl 1 mg/ml mouse IgG in PBS;
      iii. 225 μl 888 mM EDC (dilute 170.45 mg EDC in 1 ml 10 mM NaOAc buffer pH4.5);
      iv. 225 μl 222 mM NHS (dilute 25.6 mg NHS in 1 ml 10 mM NaOAc buffer pH4.5);
   e. The above 4 solutions 11 a-d were incubated in fridge at 4° C. for the required period.
12) Control solutions:
   a. 1 mg/ml T70CMD-PDEA, 200 mM EDC and 50 mM NHS solution in 10% PBS. To prepare this solution mixture were combined:
      i. 0.5 ml 2 mg/ml polymer in 10% PBS;
      ii. 50 μl 10% PBS;
      iii. 225 μl 888 mM EDC (dilute 170.45 mg EDC in 1 ml 10% PBS);
      iv. 225 μl 222 mM NHS (dilute 25.6 mg NHS in 1 ml 10% PBS);
   b. 50 μg/ml anti-biotin, 200 mM EDC and 50 mM NHS solution in 10% PBS. To prepare this solution mixture were combined:
      i. 0.5 ml 10% PBS;
      ii. 50 μl 1 mg/ml anti-biotin in PBS;
      iii. 225 μl 888 mM EDC (dilute 170.45 mg EDC in 1 ml 10% PBS);
      iv. 225 μl 222 mM NHS (dilute 25.6 mg NHS in 1 ml 10% PBS);
   c. 50 μg/ml mouse IgG, 200 mM EDC and 50 mM NHS solution in 10% PBS. To prepare this solution mixture were combined:
      i. 0.5 ml 10% PBS;
      ii. 50>1 mg/ml mouse IgG in PBS;
      iii. 225 μl 888 mM EDC (dilute 170.45 mg EDC in 1 ml 10% PBS);
      iv. 225 μl 222 mM NHS (dilute 25.6 mg NHS in 1 ml 10% PBS);
   d. The above 3 solutions were incubated in fridge at 4° C. for 1 hour.
13) 400 mM EDC in deionized water;
14) 100 mM NHS in deionized water;
15) 1 mM biotin stock solution:
   a. 10 μl of 1 mM biotin stock solution was added in 990 μl of PBS;
16) 1 mg/ml BBSA (biotinylated BSA) stock solution:
   a. 10 μl of 1 mM BBSA stock solution was added in 990 μl of PBS;

### Instrument Preparation:

### Set Up Instrument:

1. Dock desorbing chip in Biacore SPR 2000 instrument;
2. Instrument was desorbed using 0.5% SDS and 50 nM Glycine pH 9.5;
3. The desorbing chip was then undocked;
4. Dock Au-SPR chip (bare gold) in SPR instrument.

### Methods and Results

The resulting coated surfaces were then exposed to biotin or biotinylated BSA, and the response measured by SPR. The resulting traces are shown in FIG. 7.

Referring to FIG. 7, traces (i)-(iv) refer to the four different surfaces. Trace (i) is that for the surface treated with anti-biotin IgG coupled to polymer, with the attachment of the IgG/polymer complex to the surface performed in buffer at pH 7.4, (ii) is that for the surface treated at pH 7.4 with control mouse IgG coupled to polymer; (iii) is the trace for the surface treated with anti-biotin antibody/polymer, with the attachment to the surface performed in buffer at pH 4.5, and trace (iv) is that for the surface treated at pH 4.5 with control mouse IgG/polymer.

In the figure, all 4 surfaces were exposed to a 1 minute injection of biotin at 10 μM in PBS, at t=80 seconds. It is apparent from the Figure that more of the biotin bound to surface (i) [approximately a 2-fold greater response] than to the other surfaces. The biotin/anti-biotin interaction is relatively weak with fast on/off Kinetics. Accordingly, once the biotin injection ceases, bound biotin is rapidly removed by flow of buffer across the surface. After washing with buffer, a second injection of biotin was made at t=250 seconds. Again, more of the biotin bound at surface (i) than to the other surfaces. The bound biotin was again eluted by washing with buffer, and at t=430 seconds all surfaces were exposed to a 5 minute injection of 10 μg/ml biotinylated BSA. Again, the BBSA bound preferentially to surface (i). Attachment of the IgG/polymer complex to the surface at pH 7.4 clearly provides a superior surface to that where attachment was performed at pH 4.5.

### Example 4

### “One Step” Coupling of Polypeptides to Colloids

The coupling of polypeptides to colloidal suspensions of particles is extremely useful. For example colloidal gold particles, coated with monoclonal or polyclonal antibodies, are widely used in research and in diagnostic products.

In the conventional technique, several steps are involved and, at each step, the conditions (e.g. ionic strength, pH, presence and/or concentration of detergents) is needed to stop the colloidal particles aggregating as they physisorb to the polypeptide(s). In particular for the physisorption of polypeptides onto metal colloids (which are electronegative) it is necessary to perform the coupling step at a pH slightly above the isoelectric point (pI) of the polypeptide (such that the polypeptide is electropositive). In contrast, the present invention provides a method of using a free solution of polypeptide and a solution of anchoring polymer, which can be performed at a variety of different pH values.

It has the additional advantage of increasing the activity of the surface-immobilised sbp by the use of an intermediate biocompatible polymer, compared to simple physisorption, or attachment via self-assembled monolayers, that result in a higher degree of sbp inactivation, usually via surface-induced changes in conformation and the like.

In the proposed example below a solution of nanoparticles is mixed substantially simultaneously with solutions of a polymer and a sbp to achieved the desired sbp-polymer-coated nanoparticle in a single step.

### Materials

Dipotassium carbonate, Aldrich
Sodium dihydrogen phosphate, Sigma
5 M sodium chloride
Colloidal gold, 40 nm or 100 nm diameter, British Biocell International, code EM. CG40 Polyclonal antibody against E. coli O157.H7 (pAb), BacTrace, Cat. No. 01 95 50 Polymer, Dextran T70-COOH-PDEA or Dextran T70 thiosulfone

Buffer for de-salting and for initial titration work is prepared from 50 mM K₂CO₃ solution and the pH adjusted by the addition of 100 mM NaH₂PO₄ solution to give pH 8.00 at 22° C. The antibody is supplied in PBS. As a precaution, the antibodies are normally desalted to remove traces of chloride, which would otherwise threaten the integrity of the colloidal dispersion. Antibody is added onto the top of a pre-equilibrated NAP5 column containing SephadexG25 gel. The protein is eluted with the de-salt buffer, pH 8.00 into an appropriate volume of elution buffer.

Two batches of bead conjugates are prepared with the 40 nm and 100 nm colloid, using the E. coli 0157 antibodies.

Materials are combined as shown in the table below:

| Anti- E. coli | | 100 nm | Polymer at |
|---|---|---|---|
| 0157 Ab (μl) | Buffer (μl) | Gold (μl) | 1 mg/ml (μl) |
| 16.5 × 2 (33) | 4483.5 | 5000 | 500 |

Antibody is added to the buffer solution and mixed, together with the colloid solution and the polymer solution. 0.5 ml of each reaction mixture is taken and tested for stability by addition of 50 μl of 5 M NaCl solution. If no aggregation is seen, the remainder of the mixture is divided into 1.5 ml aliquots for centrifugation. The tubes are spun for 20 minutes at 12,000 rpm and 4° C., and then are turned and spun for a further 10 minutes. Most of the colloid is in a pellet in the bottom of the tubes.

The above is repeated using 40 nm colloids:

| Anti- E. coli | | 40 nm | Polymer at |
|---|---|---|---|
| 0157Ab (μl) | Buffer (μl) | Gold (μl) | 1 mg/ml (μl) |
| 3.3 | 447 | 500 | 50 |

The 40 nm conjugates are then separated by spinning at 12000 rpm in a Century chilled centrifuge in a fixed angle rotor. The colloid is divided into aliquots, so that the batch can be fitted into the rotor (microfuge holes only). After a 20 min spin, the vials are rotated 180 degrees to loosen the colloid on the walls of the tubes, and then centrifuged at 12000 rpm for a further 20 mins. The supernatants are then removed, and the pellet at the bottom of the tubes agitated in residual supernatant to re-suspend them. Sonication may also be used to re-suspended the pellet further.

The products from the 40 nm preparations are tested by dot-blotting approach on nitrocellulose membrane. Various concentrations of 0157 antigen are spotted onto the nitrocellulose and then dried. Control spots are also dotted onto the membrane, comprising various concentrations of an irrelevant antigen. The blotted membranes were then blocked by immersion in PBS containing 10% horse serum for approximately 30 mins. The membranes are then washed thoroughly with PBS, and then transferred to polypropylene sample tubes. The membranes are then incubated at 30° C. for 1 hour with a suspension of the antibody/polymer/nanoparticle complex.

The antibody/polymer/nanoparticle complex binds to the 0157 antigen dots, as indicated by appearance of a red colour, but does not bind to the control spots, showing that the binding is antigen-specific.

### Example 5

### In Situ Solution Phase Coupling

In this example, Carboxymethyl Dextran (CMD) T500 was used to produce a solution phase conjugation of anti-biotin antibody and capture of biotinylated BSA (BBSA), followed by surface inmmobilisation of the conjugate on a gold coated sensor chip. In contrast to the previous examples the activated polymer was produced “in-situ”; i.e. reagents for the attachment of the leaving group and activation of the carboxyl groups were added to polymer solution simultaneously, and in some cases the binding partner was added at the same time also. Such a procedure provides a simple, single stage conjugation reaction starting with the basic polymer.

### List of Consumables

Blank gold coated SPR chip as above,
Biacore 2000 SPR
Degassed PBS
Milli Q water
1% TritonX-100/100 mM NaOH solution (0.22 μm filtered)
EDC (Pierce 22980, lot G1100534)
NHS (Pierce 24500, lot HF106730)
Ethanolamine (1 M, pH 8.5, Akubio)
Mouse monoclonal anti-biotin Jackson ImmunoResearch 200-002-096, lot 64880 and lot 71188, bulk concentration 1.3 mg/ml
Mouse IgG, Jackson ImmunoResearch 015-000-003, Lot 67391
Biotin-BSA (BBSA), Sigma A-8549, lot 014K6070
PDEA

### Solutions Preparations

1) 0.5 mg/ml in-situ active polymer: 1.5 mg CMD500 in 100 μl 10% PBS, 11.7 mg PDEA in 520 μl 10% PBS, 13.1 mg EDC in 690 μl 10% PBS, and 7.9 mg NHS in 690 μl 10% PBS; the above solutions are mixed
2) 1 mg/ml in-situ active polymer: 1.5 mg CMD500 in 550 μl 10% PBS, 11.7 mg PDEA in 260 μl 10% PBS, 13.1 mg EDC in 345 μl 10% PBS, and 7.9 mg NHS in 345 μl 10% PBS; the above solutions are mixed
3) 1 mg/ml BSA labelled biotin (BBSA) stock solution
   a. Dissolve 1 mg BSA labelled Biotin in 1 ml M.Q. water
4) Desorb solution 1: 0.5% SDS in deionised water
5) Desorb solution 2: 50 mM Glycine pH 9.5

### Preparation of Carboxymethylated Dextran T500 (CMD-T500)

To a solution of dextran T500 (0.8 grams) in 6 M NaOH aqueous solution (10 mL), was added a solution of chloroacetic acid (5.67 grams) and sodium carbonate (3.18 grams) in 15 ml water. The resulting mixture was stirred at 95° C. for 1 hour, and then another 10 mL of 6 M NaOH and 15 mL of chloroacetic acid (5.67 grams) and sodium carbonate (3.18 grams) solutions were added. The mixture was stirred at 95° C. for a further hour. The addition of NaOH and sodium chloroacetate aqueous solutions was repeated once more, and the mixture was allowed to react for 5 hours. The reaction mixture was then acidified with concentrated HCl solution to pH 2, in an external ice-water cooling bath. The resulting polymer solution was dialyzed against MilliQ water until neutral, and lyophilized to dryness to give a white fluffy product (0.8 grams). The degree of substitution (DS) of the above CMD-T500 was determined by ¹H-NMR giving 1.22 (5.2 mmol/g).

Preparation of Polymer-Antibody Conjugate CMD-500 was coupled to anti-biotin by mixing directly the polymer and PDEA. The in-situ active T500-CMD-PDEA polymer was first coupled with anti-biotin antibody in solution under different conditions. Conditions are labelled as A-B-C in Figures X-Y, where:
A represents the anti-biotin antibody concentration in μg/ml. The same concentration of Mouse IgG was used as control for each solution;
B represents the concentration of activated polymer prepared by the above methods in mg/m; and
C represents the delay time in minutes between the preparation of in-situ active T500-CMD-PDEA polymer solution, and the addition of antibody to the mixtures. Thus, a delay of zero minutes represents simultaneous activation of the polymer and contacting with the antibody to be coupled to the activated polymer. When the delay time was 0 mins the mixture was then incubated at room temperature for 2.5 hrs before deposition in the gold coated SPR chip. When the delay time was 30 mins the mixture was incubated at room temperature for 2.0 hr before deposition on the SPR chip, as described below.

Additionally two preparations were made of the 50-0.5-30 test condition where only 50% and 10% of the PDEA reagent was added to the reaction mixture. These are shown in the Figures as 50-0.5-30-0.5, and −0.1 respectively.

After the coupling of anti-Biotin antibody or Mouse IgG with the in-situ active polymer, the blank SPR chip was coated by this mixture in solution using flow-mode (5 μl/min for 14 min) in Biacore 2000 system. When the injection was finished, the surface was post-treated by 10 mM NaOH and capped by 1M ethanolamine (pH 9.5).

### Results

A typical SPR sensorgram of flow-mode growth of in situ active T500-CMD-PDEA coupled to anti-biotin antibody (or Mouse IgG) is shown in FIG. 8.

FIG. 8 shows the results obtained for 4 different flow cells (FC1-FC4). The SPR chip was first cleaned by a 3 min injection of 100 mM NaOH/1% T-100, the 0.5 mg/ml polymer coupled with 50 μg/ml anti-biotin (30 min delay) was injected at Fc3; 0.5 mg/ml polymer coupled with 50 μg/ml irrelevant mouse IgG (0 min) was injected at Fc2; and 0.5 mg/ml polymer coupled with 50 μg/ml mouse IgG (30 min) was injected at Fc1; afterwards, the surface was post-treated by 0.5 min injection of 10 mM NaOH and capped by 7 min injection of 1M ethanolamine (pH9.5).

The coated SPR chips were then exposed to biotinylated BSA. The results are shown in FIG. 9. Starting at 72.4 seconds, there was a 5 min exposure to 10 μg/ml bBSA across all 4 flow cells. The traces for FC1 and FC3 are labelled. As is apparent, a far higher response was obtained at FC3 than at FC1, indicating specific capture of the biotinylated BSA by the anti-biotin antibody immobilised to the sensor surface. FIG. 10 shows the sensorgram for the net specific response (FC3 response minus the response from control FC1).

### Analysis of Results

The loading amount of anti-biotin coupled polymers on the SPR sensor chips and the consequent BSA labelled biotin (BBSA) binding data is summarised in FIG. 11.

In FIG. 11, the amount of polymer loaded on the test sensor surface (diagonal hatching) is shown by the scale on the right hand side, and the amount of bBSA bound by the sensors (dotted hatching) shown by the scale on the left hand side (both scales in arbitrary SPR response units). A, B and C refer to the test conditions mentioned above.

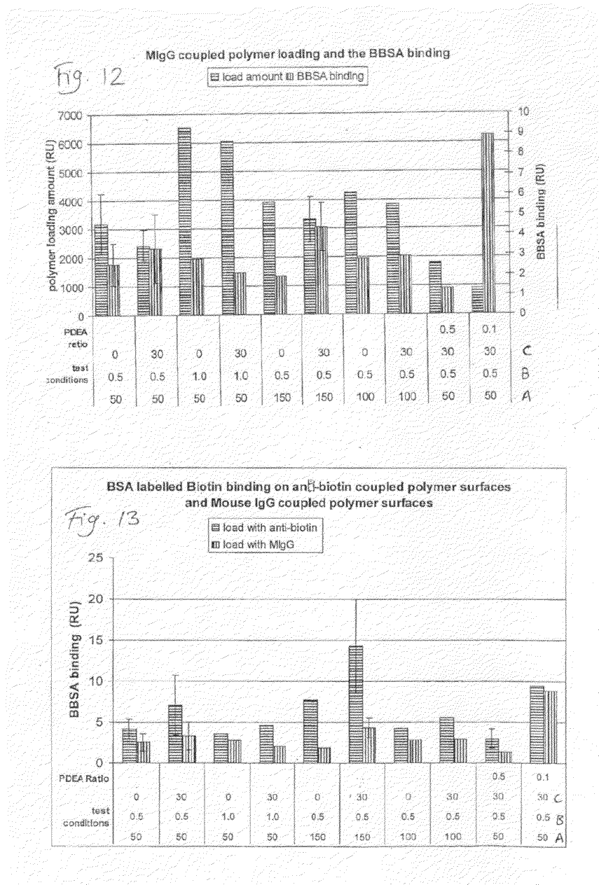

The inventors also analysed the results for the control surface, coated with CMDT500 coupled to irrelevant mouse IgG. The results are shown in FIG. 12. In FIG. 12, the amount of polymer loaded onto the sensor surface (horizontal hatching) is shown by the scale on the left hand side and the amount of bBSA bound (vertical hatching) is shown by the scale on the right hand side. Again, both scales are in arbitrary SPR response units (note especially that the scale for bBSA binding is different to the scale used in FIG. 11).

Lastly, the amount of bBSA bound on the test and control sensors was directly compared (see FIG. 13). In FIG. 13 the bBSA binding for the test sensor is shown y the bars with horizontal hatching, that for the control sensor by the bars with vertical hatching. Again the scale is arbitrary SPR response units. It is apparent that in nearly all cases the test sensor bound more bBSA than the control sensor, although in some circumstances the amount of non-specific binding (NSB) was quite high, giving low signal:noise ratios.

The better conditions out of the 10 different test conditions for specific bBSA binding are conditions: 50-0.5-5-30 and 150-0.5-30.

Comparison with NSB binding data for typical dense packed surfaces such as thiol anchored alkane chains indicates that the non-specific binding is better. The T500 based polymer also gives lower NSB than the T70 polymer. This indicates that it is possible to optimise the sample signal response over the NSB response by selection of the polymer properties such as chain length, degree of substitution and incubation period, amongst other parameters.

1. A method of attaching, indirectly, a member of a specific binding pair (or sbp) to a surface, the method comprising the steps of: (a) contacting the surface with a solution of a polymer, having side chains according to the formula X-Y-Z-R, wherein X is a spacer group; Y is a sulphur, selenium or tellurium atom; Z is a sulphur, selenium or tellurium atom, any of which may be bonded to one or two oxygen atoms;
and wherein R is any suitable moiety such that Z-R constitutes a leaving group; such that at least some of the -Z-R groups are displaced and the polymer becomes bound to the surface by X-Y groups; and (b) contacting a polymer-coated surface resulting from step (a) with a solution comprising an sbp member, so as to cause the polymer to react with the sbp member, so as to attach the sbp member, indirectly, to the surface.

2. A method of attaching, indirectly, an sbp member to a surface, the method comprising the steps of: (a) contacting, in solution, an sbp and a polymer containing side chains according to the formula X-Y-Z-R as aforesaid, so as to cause the sbp member to become attached to the polymer; and (b) contacting a solution of sbp member/polymer complex resulting from step (a) with the surface so as to cause displacement of -Z-R groups from at least some X-Y-Z-R side chains of the polymer to attach to the sbp member/polymer complex, indirectly, to the surface.

3. A method of attaching, indirectly, an sbp member to a surface, the method comprising the step of contacting the surface substantially simultaneously with both a solution of a polymer having side chains according to the formula X-Y-Z-R as aforesaid; and a solution of an sbp member.

4. A method according to claim 1 wherein the solution of polymer is an aqueous solution.

5. A method according to claim 4 wherein the solution of sbp member is an aqueous solution.

6. A method according to claim 4, wherein the sbp member is attached to the polymer by reaction with at least some of the X-Y-Z-R side chains of the polymer so as to form a side chain X-Y-Z-R₁, wherein R₁ is the member of the sbp.

7. A method according to claim 4, wherein the sbp member is attached to the polymer by reaction with a reactive group on the polymer, which reactive group may or may not form part of the X-Y-Z-R side chain, and wherein the reactive group is selected from: hydroxy, carboxy, amino, or epoxy groups.

8. A method according to claim 4, wherein the surface comprises a metal selected from the group consisting of: gold, silver, platinum, palladium, nickel, chromium, titanium, copper, and any alloy thereof.

9. A method according to claim 4, wherein the surface is essentially planar.

10. A method according to claim 4, wherein the surface comprises a colloidal suspension of particles.

11. A method according to claim 10, wherein the surface comprises a suspension of colloidal gold or quantum dots.

12. A method according to claim 4, wherein the polymer is a polysaccharide, optionally derivatised.

13. A method according to claim 12, wherein the polymer comprises dextran, cellulose, agarose or sepharose, any of which may optionally be derivatised.

14. A method according to claim 4, wherein the polymer has an average molecular weight in the range 10 KDa-1,000 KDa.

15. A method according to claim 4, wherein the polymer has an average molecular weight in the range 50 KDa-1,000 KDa.

16. A method according to claim 4, wherein the polymer has an average molecular weight in the range 100 KDa-1,000 KDa.

17. A method according to claim 4, wherein the polymer has an average molecular weight in the range 250 KDa-1,000 KDa.

18. A method according to claim 4, wherein the member of the sbp is selected from the group consisting of: antibodies, antigens, ligands, receptors, and complementary nucleotide sequences.

19. A method according to claim 4, wherein the member of the sbp comprises a polypeptide.

20. A method according to claim 4, wherein the member of the sbp is an antibody or an antigen-binding fragment of an antibody.

21. A method according to claim 4, wherein the surface forms part of a component for use in a biosensor.

22. A method according to claim 1, comprising the steps of:
(a) converting a base polymer into a derivatised polymer comprising X-Y-Z-R side chains;
(b) optionally, activating one or more reactive groups present in the polymer;
(c) coupling the sbp member to the derivatized polymer to form an sbp member/polymer complex; and
(d) attaching the sbp member/polymer complex to the surface.

23. A method according to claim 1, comprising the steps of:
(a) converting a base polymer into a derivatised polymer comprising X-Y-Z-R side chains;
(b) optionally, activating one or more reactive groups present in the polymer;
(c) attaching the derivatised polymer to the surface; and
(d) coupling the sbp member to the surface-bound derivatised polymer.

24. A method according to claim 22, wherein two or more of steps (a)-(c) are performed substantially simultaneously.

25. A method according to claim 23, wherein each of steps (a)-(c) are performed substantially simultaneously.

26. A method according to claim 4, wherein a base polymer is converted into a derivatised polymer comprising X-Y-Z-R side chains by use of one or more reagents comprising 2-(pyridinyldithio)ethaneamine (“PDEA”).

27. A method according to claim 22, wherein the reactive groups are activated by reaction with reagents comprising N-ethyl-N¹-(3-dimethyl-amino-propyl)-carbodiimide hydrochloride (“EDC”) and N-hydroxysuccinimide (“NHS”).

28. A method according to claim 4, wherein the method is substantially performed in a single reaction vessel.

29. A surface coated with a complex of polymer/sbp member, prepared by the method of claim 4.

30. (canceled)
